(19) United States
(12) Patent Application Publication
(10) Pub. No.: US 2021/0042916 A1
(43) Pub. Date: Feb. 11, 2021

(54) DEEP LEARNING-BASED DIAGNOSIS AND REFERRAL OF DISEASES AND DISORDERS

(71) Applicants: AI TECHNOLOGIES INC., Grand Cayman (KY); THE REGENTS OF THE UNIVERSITY OF CALIFORNIA, Oakland, CA (US)

(72) Inventors: Kang ZHANG, San Diego, CA (US); Rui HOU, Shenyang (CN); Lianghong ZHENG, Shenyang (CN)

(21) Appl. No.: 16/968,520

(22) PCT Filed: Feb. 7, 2019
(86) PCT No.: PCT/US2019/017101

§ 371 Date: Aug. 7, 2020

Related U.S. Application Data
(60) Provisional application No. 62/627,605, filed on Feb. 7, 2018.

(51) Int. Cl. G06T 7/00; G06N 3/08; G06N 3/04; G16H 50/20; G16H 30/40; G16H 50/70; A61B 6/00

(52) U.S. Cl. CPC G06T 7/0012; G06N 3/08; G06N 3/04; G16H 50/20; G16H 30/40; G06T 2207/20084; A61B 6/50; G06T 2207/30061; G06T 2207/20081; G06T 2207/10116; G16H 50/70

(57) ABSTRACT

Disclosed herein are systems, methods, devices, and media for carrying out medical diagnosis of diseases and conditions using artificial intelligence or machine learning approaches. Deep learning algorithms enable the automated analysis of medical images such as X-rays to generate predictions of comparable accuracy to clinical experts for various diseases and conditions including those afflicting the lung such as pneumonia.

### CROSS-REFERENCE

This application claims the benefit of U.S. Provisional Application No. 62/627,605, filed Feb. 7, 2018, which is incorporated herein by reference in its entirety.

### BACKGROUND OF THE DISCLOSURE

Many lung diseases and disorders are diagnosed based on medical imaging. Medical imaging has traditionally relied upon human experts to analyze images individually. As the number of medical imaging procedures increase, demand for efficient and accurate image analysis is outstripping the supply of experts capable of performing this function.

### SUMMARY OF THE DISCLOSURE

Traditional algorithmic approaches to medical image analysis suffer from numerous technical deficiencies related to an inability to adequately perform the analysis without significant human intervention and/or guidance, which belies the supposed promise of artificial intelligence and machine learning to revolutionize disease diagnosis and management. For example, one approach relies upon (1) handcrafted object segmentation, (2) identification of each segmented object using statistical classifiers or shallow neural computational machine-learning classifiers designed specifically for each class of objects, and (3) classification of the image. As a result, the creation and refinement of multiple classifiers required considerable expertise and time, and was computationally expensive. In addition, the training of machine learning classifiers is often deficient due to a lack of sufficient medical images in the training set. This problem is exacerbated in the case of diseases or conditions that are relatively rare or lack adequate access to the medical images. Moreover, because machine learning often behaves like a black box, acceptance of diagnoses generated through such methods can be hindered due to the lack of transparency on how the classifier evaluates a medical image to generate a prediction.

The present disclosure solves these technical problems with existing computer systems carrying out image analysis by providing improved systems and techniques that do not require substantial intervention by an expert to generate the classifiers. These include, for example, convolutional neural network layers that provide multiple processing layers to which image analysis filters or convolutions are applied. The abstracted representation of images within each layer is constructed by systematically convolving multiple filters across the image to produce a feature map used as input for the following layer. This overall architecture enables images to be processed into pixels as input and to generate the desired classification as output. Accordingly, the multiple resource-intensive steps used in traditional image analysis techniques such as handcrafted object segmentation, identification of the segmented objects using a shallow classifier, and classification of the image is no longer required.

In addition, the present disclosure solves the technical problem of insufficient images in the relevant domain (e.g., medical images for a specific lung disease) for training algorithms to effectively perform image analysis and/or diagnosis. Certain embodiments of the present disclosure include systems and techniques applying a transfer learning algorithm to train an initial machine learning algorithm such as a convolutional neural network on images outside of the specific domain of interest to optimize the weights in the lower layer(s) for recognizing the structures found in the images. The weights for the lower layer(s) are then frozen, while the weights of the upper layer(s) are retrained using images from the relevant domain to identify output according to the desired diagnosis (e.g., identification or prediction of specific diseases or conditions). This approach allows the classifier to recognize distinguishing features of specific categories of images (e.g., X-ray images of the lung or chest cavity) far more quickly using significantly fewer training images and while requiring substantially less computational power. The use of non-domain images to partially train or pre-train the classifier allows optimization of the weights of one or more of the neural network layers using a deep reservoir of available images corresponding to thousands of categories. The result is a classifier having a sensitivity, specificity, and accuracy that is unexpected and surprising compared to the traditional approach, especially in view of the improvements in speed, efficiency, and computational power required. Indeed, certain embodiments of the classifier outperform human experts in correctly diagnosing medical images according to sensitivity, specificity, accuracy, or a combination thereof.

The present disclosure also addresses the black box nature of machine learning by allowing identification of the critical areas contributing most to the classifier's predicted diagnosis. Certain embodiments of the present disclosure utilize occlusion testing on test images to identify the regions of interest that contribute the highest importance to the classifier's ability to generate accurate diagnoses. These regions can be verified by experts to validate the system, which creates greater transparent and increases trust in the diagnosis.

The technological solutions to the technological problem of effectively implementing computer-based algorithmic image analysis described herein opens up the previously unrealized potential of machine learning techniques to revolutionize medical image analysis and diagnosis. Furthermore, the present disclosure provides additional technical advantages over existing computer systems and techniques that are described in more detail below.

In certain embodiments, disclosed herein is a method for providing a medical diagnosis, comprising: a) obtaining a medical image of a lung; b) evaluating the medical image using a predictive model trained using a machine learning procedure; and c) determining, by the predictive model, whether or not the medical image is indicative of a disease or disorder of the lung, the determination having a sensitivity greater than 90% and a specificity greater than 90%. In some embodiments, the machine learning procedure comprises a deep learning procedure. In some embodiments, the machine learning procedure comprises a convolutional neural network. In some embodiments, the method further comprises subjecting the medical image of the lung to an image occlusion procedure. In some embodiments, the machine learning procedure comprises a transfer learning procedure. In some embodiments, the transfer learning procedure comprises pre-training the machine learning procedure using non-medical or unlabeled medical images obtained from a large image dataset to obtain a pre-trained model. In some embodiments, the transfer learning procedure further comprises training the pre-trained model using a set of medical images that is smaller than the large image dataset. In some embodiments, the method further comprises making a medical treatment recommendation based on the determination. In some embodiments, the medical image of the lung is a chest X-ray. In some embodiments, the disease or disorder of the lung is selected from the group consisting of: pneumonia, childhood pneumonia, emphysema, tuberculosis, and lung cancer. In some embodiments, the system further comprises an imaging device in operative communication with the digital processing device. In some embodiments, the determination is made by uploading the image to a cloud for remote analysis and receiving the determination generated by the cloud. In some embodiments, the transfer learning procedure comprises pre-training a first model on a first image data set, freezing at least a portion of the first model, generating a second model comprising the at least a portion of the first model, and training the second model on a second image data set labeled with a diagnostic status of the disease or condition.

In certain embodiments, disclosed herein is non-transitory computer-readable medium comprising machine-executable code that, upon execution by one or more computer processors, implements a method for providing a medical diagnosis, the method comprising: a) obtaining a medical image of a lung; b) evaluating the medical image using a predictive model trained using a machine learning procedure; and c) determining, by the predictive model, whether or not the medical image is indicative of a disease or disorder of the lung, the determination having a sensitivity greater than 90% and a specificity greater than 90%. In some embodiments, the machine learning procedure comprises a deep learning procedure. In some embodiments, the machine learning procedure comprises a convolutional neural network. In some embodiments, the method further comprises subjecting the medical image of the lung to an image occlusion procedure. In some embodiments, the machine learning procedure comprises a transfer learning procedure. In some embodiments, the transfer learning procedure comprises pre-training the machine learning procedure using non-medical or unlabeled medical images obtained from a large image dataset to obtain a pre-trained model. In some embodiments, the transfer learning procedure further comprises training the pre-trained model using a set of medical images that is smaller than the large image dataset. In some embodiments, the method further comprises making a medical treatment recommendation based on the determination. In some embodiments, the medical image of the lung is a chest X-ray. In some embodiments, the disease or disorder of the lung is selected from the group consisting of: pneumonia, childhood pneumonia, emphysema, tuberculosis, and lung cancer. In some embodiments, the system further comprises an imaging device in operative communication with the digital processing device. In some embodiments, the determination is made by uploading the image to a cloud for remote analysis and receiving the determination generated by the cloud. In some embodiments, the transfer learning procedure comprises pre-training a first model on a first image data set, freezing at least a portion of the first model, generating a second model comprising the at least a portion of the first model, and training the second model on a second image data set labeled with a diagnostic status of the disease or condition.

In certain embodiments, disclosed herein is a computer-implemented system comprising: a digital processing device comprising: at least one processor, an operating system configured to perform executable instructions, a memory, and a computer program including instructions executable by the digital processing device to create an application for providing a medical diagnosis of a disease or disorder or a lung, the application comprising: a) a software module for obtaining a medical image of a lung; b) a software module for analyzing the medical image using a predictive model trained using a machine learning procedure; and c) a software module for determining, by the predictive model, whether or not the medical image of the lung is indicative of a disease or disorder of the lung, the determination having a sensitivity greater than 90% and a specificity greater than 90%. In some embodiments, the machine learning procedure comprises a deep learning procedure. In some embodiments, the machine learning procedure comprises a convolutional neural network. In some embodiments, the application further comprises a software module for subjecting the medical image of the lung to an image occlusion procedure. In some embodiments, the machine learning procedure comprises a transfer learning procedure. In some embodiments, the transfer learning procedure comprises pre-training the machine learning procedure using non-domain medical images obtained from a large image dataset to obtain a pre-trained model. In some embodiments, the transfer learning procedure further comprises training the pre-trained model using a set of labeled medical images that is smaller than the large image dataset. In some embodiments, the application further comprises a software module for making a medical treatment recommendation based on the determination. In some embodiments, the medical image of the lung is a chest X-ray. In some embodiments, the disease or disorder of the lung is selected from the group consisting of: pneumonia, childhood pneumonia, emphysema, tuberculosis, and lung cancer. In some embodiments, the system further comprises an imaging device in operative communication with the digital processing device. In some embodiments, the determination is made by uploading the image to a cloud for remote analysis and receiving the determination generated by the cloud. In some embodiments, the transfer learning procedure comprises pre-training a first model on a first image data set, freezing at least a portion of the first model, generating a second model comprising the at least a portion of the first model, and training the second model on a second image data set labeled with a diagnostic status of the disease or condition.

In certain embodiments, the present disclosure relates to a method for providing a medical diagnosis, the method comprises: obtaining a medical image of a lung; performing a machine learning procedure on the medical image of the lung; and determining, by the machine learning procedure, whether or not the medical image is indicative of a disease or disorder of the lung, the determination having a sensitivity greater than 90% and a specificity greater than 90%. In some non-limiting embodiments, the machine learning procedure comprises a deep learning procedure. In some non-limiting embodiments, the machine learning procedure comprises a convolutional neural network. In some non-limiting embodiments, the method further comprises subjecting the medical image of the lung to an image occlusion procedure. In some non-limiting embodiments, the method further comprises performing a transfer learning procedure. In some non-limiting embodiments, the transfer learning procedure comprises pre-training the machine learning procedure using non-medical images obtained from a large image dataset to obtain a pre-trained machine learning procedure. In some non-limiting embodiments, the transfer learning procedure comprises pre-training the machine learning procedure using non-domain or unlabeled or undiagnosed medical images obtained from a large image dataset to obtain a pre-trained machine learning procedure. In some non-limiting embodiments, the transfer learning procedure further comprises training the pre-trained machine learning procedure using a set of medical images that is smaller than the large image dataset. In some non-limiting embodiments, the transfer learning procedure further comprises training the pre-trained machine learning procedure using a set of labeled or diagnosed medical images that is smaller than the large image dataset. In some non-limiting embodiments, the method further comprises making a medical treatment recommendation based on the determination. In some non-limiting embodiments, the medical image of the lung is a chest X-ray. In some non-limiting embodiments, the medical disorder is selected from the group consisting of: pneumonia, childhood pneumonia, emphysema, and lung cancer. In some embodiments, the system further comprises an imaging device in operative communication with the digital processing device. In some embodiments, the determination is made by uploading the image to a cloud for remote analysis and receiving the determination generated by the cloud. In some embodiments, the transfer learning procedure comprises pre-training a first model on a first image data set, freezing at least a portion of the first model, generating a second model comprising the at least a portion of the first model, and training the second model on a second image data set labeled with a diagnostic status of the disease or condition.

In certain embodiments, the present disclosure relates to a non-transitory computer-readable medium comprising machine-executable code that, upon execution by one or more computer processors, implements a method for providing a medical diagnosis, the method comprises: obtaining a medical image of a lung; performing a machine learning procedure on the medical image of the lung; and determining, by the machine learning procedure, whether or not the medical image is indicative of a disease or disorder of the lung, the determination having a sensitivity greater than 90% and a specificity greater than 90%. In some non-limiting embodiments, the machine learning procedure comprises a deep learning procedure. In some non-limiting embodiments, the machine learning procedure comprises a convolutional neural network. In some non-limiting embodiments, the method further comprises subjecting the medical image of the lung to an image occlusion procedure. In some non-limiting embodiments, the method further comprises performing a transfer learning procedure. In some non-limiting embodiments, the transfer learning procedure comprises pre-training the machine learning procedure using non-medical images obtained from a large image dataset to obtain a pre-trained machine learning procedure. In some non-limiting embodiments, the transfer learning procedure further comprises training the pre-trained machine learning procedure using a set of medical images that is smaller than the large image dataset. In some non-limiting embodiments, the method further comprises making a medical treatment recommendation based on the determination. In some non-limiting embodiments, the medical image of the lung is a chest X-ray. In some non-limiting embodiments, the medical disorder is selected from the group consisting of: pneumonia, childhood pneumonia, emphysema, and lung cancer. In some embodiments, the system further comprises an imaging device in operative communication with the digital processing device. In some embodiments, the determination is made by uploading the image to a cloud for remote analysis and receiving the determination generated by the cloud. In some embodiments, the transfer learning procedure comprises pre-training a first model on a first image data set, freezing at least a portion of the first model, generating a second model comprising the at least a portion of the first model, and training the second model on a second image data set labeled with a diagnostic status of the disease or condition.

In certain embodiments, the present disclosure relates to a computer-implemented system comprising: a digital processing device comprising: at least one processor, an operating system configured to perform executable instructions, a memory, and a computer program including instructions executable by the digital processing device to create an application for providing a medical diagnosis, the application comprising: a software module for obtaining a medical image of a lung; a software module for performing a machine learning procedure on the medical image of the lung; and a software module for determining, by the machine learning procedure, whether or not the medical image is indicative of a medical disease or disorder of the lung, the determination having a sensitivity greater than 90% and a specificity greater than 90%. In some non-limiting embodiments, the machine learning procedure comprises a deep learning procedure. In some non-limiting embodiments, the machine learning procedure comprises a convolutional neural network. In some non-limiting embodiments, the application further comprises a software module for subjecting the medical image of the lung to an image occlusion procedure. In some non-limiting embodiments, the application further comprises a software module for performing a transfer learning procedure. In some non-limiting embodiments, the transfer learning procedure comprises pre-training the machine learning procedure using non-medical images obtained from a large image dataset to obtain a pre-trained machine learning procedure. In some non-limiting embodiments, the transfer learning procedure further comprises training the pre-trained machine learning procedure using a set of medical images that is smaller than the large image dataset. In some non-limiting embodiments, the application further comprises a software module for making a medical treatment recommendation based on the determination. In some non-limiting embodiments, the medical image of the lung is a chest X-ray. In some non-limiting embodiments, the medical disorder is selected from the group consisting of: pneumonia, childhood pneumonia, emphysema, and lung cancer. In some embodiments, the system further comprises an imaging device in operative communication with the digital processing device. In some embodiments, the determination is made by uploading the image to a cloud for remote analysis and receiving the determination generated by the cloud. In some embodiments, the transfer learning procedure comprises pre-training a first model on a first image data set, freezing at least a portion of the first model, generating a second model comprising the at least a portion of the first model, and training the second model on a second image data set labeled with a diagnostic status of the disease or condition.

### BRIEF DESCRIPTION OF THE DRAWINGS

A better understanding of the features and advantages of the present invention will be obtained by reference to the following detailed description that sets forth illustrative embodiments, in which the principles of the invention are utilized, and the accompanying drawings of which:

FIG. 1 shows illustrative examples of chest x-rays in patients with pneumonia. The normal chest x-ray (left panel) depicts clear lungs without any areas of abnormal opacification in the image. Bacterial pneumonia (middle) typically exhibits a focal lobar consolidation, in this case in the right upper lobe (white arrows), whereas viral pneumonia (right) manifests with a more diffuse “interstitial” pattern in both lungs.

FIG. 2 shows plots depicting performance of pneumonia diagnosis using chest x-ray images in the training (orange) and validation (blue) datasets using TensorBoard. Comparisons were made for pneumonia versus normal (A) with cross-entropy loss plotted against the training step (B), as well as comparisons between bacterial pneumonia and viral pneumonia (C) and the associated cross-entropy loss (D). Plots were normalized with a smoothing factor of 0.6 in order to clearly visualize trends. The area under the receiver operating characteristic (ROC) curve for detecting pneumonia versus normal was 98.1% (E). The area under the ROC curve for detecting bacterial versus viral pneumonia was 95.0% (F).

### DETAILED DESCRIPTION OF THE DISCLOSURE

It is recognized that implementation of clinical decision support algorithms for medical imaging with improved reliability and clinical interpretability can be achieved through one or combinations of technical features of the present disclosure. According to some aspects of the present disclosure, disclosed herein is a diagnostic tool to analyze medical imaging by presenting a deep learning framework developed for patients with common and treatable diseases or disorders of the lung. In some embodiments, the disclosed framework implements a transfer learning algorithm, which allows for the training of a highly accurate neural network with a fraction of the data required in more conventional approaches. In some embodiments, the model disclosed herein generalizes and performs well on many medical classification tasks. In some instance, multiple imaging modalities are desired in order to reliably and accurately diagnose all the different diseases or disorders of the lung, and the approach disclosed in some embodiments yields state-of-the-art performance across many imaging techniques. Certain embodiments of this approach yield superior performance across many imaging techniques.

In some embodiments, this machine learning approach is applied to a large and clinically heterogeneous dataset of x-ray images and is capable of achieving diagnostic performance that is comparable to or superior to that of human experts in classifying diseases or conditions such as pneumonia or childhood pneumonia. In some embodiments, the algorithms disclosed herein provide a more transparent and interpretable diagnosis, compared to traditional deep learning algorithms, by using image occlusion to highlight clinically significant regions within images as understood by the neural network. Furthermore, certain embodiments of the transfer learning approach scales with additional training images and development of clinical imaging datasets as well as with continuing advancements in the field of convolutional neural networks (CNN) and image processing. In some embodiments, provided herein is a platform that interfaces with web and/or mobile applications that upload medical images for remote diagnosis with high accuracy. The algorithm not only demonstrates strong performance for lung disease, but also holds broad clinical utility for image-based diagnosis of other diseases.

It is recognized in the present disclosure that Artificial intelligence (AI) has the potential to revolutionize disease diagnosis and healthcare management by performing classification currently difficult for human experts and by rapidly reviewing immense amounts of imaging data. Despite its potential, clinical interpretability and feasible preparation of the AI remain challenging.

Traditional image analysis often relied on handcrafted object segmentation followed by identification of each object with shallow machine learning classifiers designed specifically for each class of objects. Creating and refining multiple classifiers required many skilled people and much time. The multiple steps required of a mature analyzing system to classify an image were computationally expensive. Deep learning networks (DNNs) provide a revolutionary step forward in machine learning technique because DNN classifiers subsume the complex steps that previously needed to be handcrafted to generate a diagnosis from an image. As a result, in various embodiments, a trained DNN classifies a medical image in significantly less time than a human.

In some embodiments, automated recognition systems are developed using a limited amount of image data. With the advent of smartphones and digital cameras, the growth in image data has been exponential. This explosion of data and its widespread availability on the web have led to a need for effective methods for analyzing the huge amount of data efficiently without time-consuming and complex steps. As disclosed herein, DNNs make it possible to analyze the large amount of data currently being generated, and likewise, the large amount of data make it possible for DNNs to be well trained.

As disclosed herein, in certain embodiments, convolutional neural network (CNN) layers allow for significant gains in the ability to classify images and detect objects in a picture. In various embodiments, CNNs are composed of multiple processing layers to which image analysis filters, or convolutions, are applied. In some embodiments, the abstracted representation of images within each layer is constructed by systematically convolving multiple filters across the image, producing a feature map which is used as input to the following layer. CNNs learn representations of images with multiple levels of increasing understanding of the image contents, which is what makes the networks deep. This deep learning method is capable of discovering intricate structures in large data sets by using the backpropagation learning algorithm to change its internal parameters to minimize errors in making the desired classification. Each layer is increasingly sophisticated in its representation of the organization of the data compared to the previous layer. The first few layers of the neural network can extract simple structures, such as lines and edges, while the layers up the chain begin to determine more complex structures. This architecture makes it possible to process images in the form of pixels as input and to give the desired classification as output. Accordingly, in certain embodiments, the image-to-classification approach in one classifier replaces the multiple steps of previous image analysis methods. As a result, the CNNs disclosed herein dramatically improve the state-of-the-art in visual object recognition.

Disclosed herein, in certain aspects, are methods of addressing a lack of data in a given domain by leveraging data from a similar domain. For example, a large database of labeled images has been collected and made available as ImageNet with 1000 object categories. In certain embodiments, a CNN is first trained on this dataset to develop features at its lower layers that are important for discriminating objects. In further embodiments, a second network is created that copies the parameters and structure of the first network, but with the final layer(s) optionally re-structured as needed for a new task. In certain embodiments, these final layer(s) are configured to perform the classification of lung images. Thus, in some embodiments, the second network uses the first network to seed its structure. This allows training to continue on the new, but related task. In some embodiments, the first network is trained using labeled images comprising non-domain images (e.g., images not labeled with the classification), and the second network is trained using labeled images comprising domain images (e.g., classified images) to complete the training allowing for high accuracy diagnosis of lung disorders and/or conditions. The method of transferring general classification knowledge from one domain to another is called transfer learning. As disclosed herein, the application of transfer learning within the field of machine learning-based diagnosis of diseases and conditions has proven to be a highly effective technique, particularly when faced with domains with limited data. By retraining a model with weights already optimized to recognize the features of standard objects rather than training a completely blank network, the model or classifier can recognize the distinguishing features of images much faster and with significantly fewer training examples.

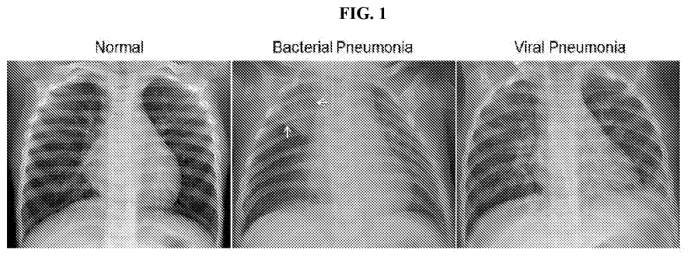

Disclosed herein, in certain embodiments, is a transfer learning algorithm for analyzing x-ray images for the diagnosis of common causes of lung diseases. According to the World Health Organization (WHO), pneumonia kills about 2 million children under 5 years old every year, and is consistently estimated as the single leading cause of childhood mortality (Rudan et al., 2008), killing more children than HIV/AIDS, malaria, and measles combined (Adegbola, 2012). The WHO reports that nearly all cases (95%) of new onset childhood clinical pneumonia occur in developing countries, particularly in Southeast Asia and Africa. Bacterial and viral pathogens are the two leading causes of pneumonia (Mcluckie, 2009) but require very different forms of management. Bacterial pneumonia requires urgent referral for immediate antibiotic treatment, while viral pneumonia is treated with supportive care. Therefore, accurate and timely diagnosis is imperative. One key element of diagnosis is radiographic data, since chest x-rays are routinely obtained as standard of care and can help differentiate between different types of pneumonia (FIG. 1). However, rapid radiologic interpretation of images is not always available, particularly in the low-resource settings where childhood pneumonia has the highest incidence and highest rates of mortality. Accordingly, provided herein is a transfer learning framework for training a classifier to in classify pediatric chest x-rays to detect pneumonia and furthermore to distinguish viral and bacterial pneumonia to facilitate rapid referrals for children needing urgent intervention.

In some embodiments, the transfer learning algorithm is applied to a small sample of chest x-rays in order to evaluate the preliminary performance on distinguishing between different types of pneumonia such as bacterial pneumonia and viral pneumonia. Distinguishing the chest x-rays is challenging because rather than separating a normal state from diseased ones, this scenario entails distinguishing between two disease states with subtle differences.

Another advantage of the present disclosure is the use of an AI model as a triage system to generate a referral, mimicking real-world applications in community settings, primary care, and urgent care clinics. These embodiments may ultimately confer broad public health impact by promoting earlier diagnosis and detection of disease progression, thereby facilitating treatment that can improve outcomes and quality of life.

According to one aspect of the present disclosure, a general AI platform for diagnosis and referral of two common lung diseases: pneumonia and childhood pneumonia. By employing a transfer learning algorithm, a model according to the methods disclosed herein demonstrated competitive performance of x-ray image analysis without the need for a highly specialized deep learning machine and without a database of millions of example images. Moreover, the model's performance in diagnosing lung x-ray images was comparable to that of human experts with significant clinical experience with lung diseases. When the model was trained with a much smaller number of images (about 1000 from each class), its accuracy, sensitivity, specificity, and area under the ROC curve were all slightly decreased compared with the model trained on over 150,000 total images, but it was still overall a very high-performing system, thereby illustrating the power of the transfer learning system to make highly effective classifications even with a very limited training dataset.

In some embodiments, a predictive model generated according to the methods described herein is assessed for one or more performance metrics, optionally in comparison to human experts or experienced clinicians (e.g., radiologists).

According to one aspect of the present disclosure, an occlusion test to identify the areas of greatest importance used by the model in assigning diagnosis is performed. The greatest benefit of an occlusion test is that it reveals insights into the decisions of neural networks, which are sometimes referred to as “black boxes” with no transparency. Since this test is performed after training is completed, it demystifies the algorithm without affecting its results. The occlusion test also confirms that the network makes its decisions using accurate distinguishing features.

Although transfer learning allows the training of a highly accurate model with a relatively small training dataset, its performance would be inferior to that of a model trained from a random initialization on an extremely large dataset of x-ray images, since even the internal weights can be directly optimized for x-ray feature detection. However, transfer learning using a pre-trained model trained on millions of various medical images would likely yield a more accurate model when retraining layers for other medical classifications.

The performance of the model depends highly on the weights of the pre-trained model. Therefore, in some embodiments, the performance of this model is enhanced when tested on a larger ImageNet dataset with more advanced deep learning techniques and architecture. Further, the rapid progression and development of the field of convolutional neural networks applied outside of medical imaging would also improve the performance of this approach.

In some embodiments, x-ray imaging is used as a demonstration of a generalized approach in medical image interpretation and subsequent decision making. The disclosed framework identified potential pathology on a tissue map to make a referral decision with performance comparable to human experts, enabling timely diagnosis of two common lung disorders. In order to tackle the reproducibility and transparency issues brought on by training and testing on a protected or proprietary dataset, such as medical x-ray imagery, an easy-to-use tool was generated that allows testing of this model on any provided x-ray image. This tool simply loads the trained model and predicts the diagnosis of any user-provided image with a breakdown using softmax probabilities. This application allows anyone with access to it the ability to test this algorithm and even upload smartphone captures of x-ray images and yield comparable accuracy. A public version of the tool has also been made available at https://www.medfirstview.com with the most accurate model to demonstrate the performance of this deep learning approach.

Furthermore, the disclosed network represents a generalized platform which in some embodiments is apply to medical imaging techniques other than x-ray (e.g., MRI, CT, etc.) to make a clinical diagnostic decision. In some embodiment, the CT image is a cross-sectional image of a CT scan. Without wishing to be bound by any particular theory, the use of the platform technology described herein facilitates screening programs and allows more efficient referral systems, particularly in remote or low-resource areas, leading to a broad clinical and public health impact.

### Medical Imaging

In certain aspects, the machine learning framework disclosed herein is used for analyzing medical imaging data. In some embodiments, the medical imaging data comprises radiological images, which can include images of chest cavity. The framework described herein is applicable to various types of medical imaging including X-rays. X-rays include chest X-rays, lung X-rays, abdomen X-rays, and KUB X-rays (kidney, ureter, bladder X-ray). Medical images can also include MRIs, CT scans, and other relevant medical imaging.

A lack of sufficient suitable medical images or medical imaging data can lead to inaccurate or poorly trained classifiers. However, embodiments of the systems, methods, and devices disclosed herein implement transfer learning to improve the training of models using images or imaging data that is not suitable for directly training the classifier. In some embodiments, a model is trained during a first step using a first set of images. In some embodiments, transfer learning is implemented to further train a model on suitable medical images (e.g., X-ray images labeled with diagnostic outcomes). By leveraging additional images that are not labeled for the diagnostic outcome for part of the training, a trained model or classifier can be generated that provides improved predictive accuracy compared to a model trained using only the available labeled medical images, which may form a small data set.

In some embodiments, the algorithms disclosed herein such as machine learning algorithms use transfer learning. In some embodiments, the algorithms disclosed herein use images to pre-train a model or classifier. In some embodiments, the algorithms disclosed herein achieve at least one performance metric (an accuracy, sensitivity, specificity, AUC, positive predictive value, negative predictive value, or any combination thereof) for an independent data set (e.g., test dataset not used in training) that is at least 80%, 85%, 90%, 91%, 92%, 93%, 94%, 95%, 96%, 97%, 98%, or at least 99% similar to an algorithm that is trained using labeled medical images alone. In some embodiments, the similar performance metric is obtained when the transfer learning procedure and the non-transfer learning procedure utilize the same set of medical images for training. In some embodiments, transfer learning provides a model that performs better than a model generated using the same labeled data set without transfer learning.

In some embodiments, a machine learning algorithm or model is trained using medical images numbering about 50 to about 50,000. In some embodiments, a machine learning algorithm or model is trained using medical images numbering at least about 50. In some embodiments, a machine learning algorithm or model is trained using medical images numbering at most about 50,000. In some embodiments, a machine learning algorithm or model is trained using medical images numbering about 50 to about 100, about 50 to about 200, about 50 to about 300, about 50 to about 400, about 50 to about 500, about 50 to about 1,000, about 50 to about 5,000, about 50 to about 10,000, about 50 to about 20,000, about 50 to about 30,000, about 50 to about 50,000, about 100 to about 200, about 100 to about 300, about 100 to about 400, about 100 to about 500, about 100 to about 1,000, about 100 to about 5,000, about 100 to about 10,000, about 100 to about 20,000, about 100 to about 30,000, about 100 to about 50,000, about 200 to about 300, about 200 to about 400, about 200 to about 500, about 200 to about 1,000, about 200 to about 5,000, about 200 to about 10,000, about 200 to about 20,000, about 200 to about 30,000, about 200 to about 50,000, about 300 to about 400, about 300 to about 500, about 300 to about 1,000, about 300 to about 5,000, about 300 to about 10,000, about 300 to about 20,000, about 300 to about 30,000, about 300 to about 50,000, about 400 to about 500, about 400 to about 1,000, about 400 to about 5,000, about 400 to about 10,000, about 400 to about 20,000, about 400 to about 30,000, about 400 to about 50,000, about 500 to about 1,000, about 500 to about 5,000, about 500 to about 10,000, about 500 to about 20,000, about 500 to about 30,000, about 500 to about 50,000, about 1,000 to about 5,000, about 1,000 to about 10,000, about 1,000 to about 20,000, about 1,000 to about 30,000, about 1,000 to about 50,000, about 5,000 to about 10,000, about 5,000 to about 20,000, about 5,000 to about 30,000, about 5,000 to about 50,000, about 10,000 to about 20,000, about 10,000 to about 30,000, about 10,000 to about 50,000, about 20,000 to about 30,000, about 20,000 to about 50,000, or about 30,000 to about 50,000. In some embodiments, a machine learning algorithm or model is trained using medical images numbering about 50, about 100, about 200, about 300, about 400, about 500, about 1,000, about 5,000, about 10,000, about 20,000, about 30,000, or about 50,000.

### Machine Learning

Disclosed herein, in various embodiments, are machine learning methods for analyzing medical data including, for example, X-ray images. In an exemplary embodiment, the machine learning framework disclosed herein is used for analyzing X-ray images for the diagnosis of diseases or conditions detectable by X-ray images. In some cases, the X-ray images are analyzed to detect lung diseases or conditions. Examples of lung diseases and conditions include chronic obstructive pulmonary disease, cystic fibrosis, lung cancer, pneumonia, interstitial lung disease, hiatal hernia, and pneumothorax. In some embodiments, the X-ray image is used to detect a heart condition such as heart failure. In some embodiments, the detection or diagnosis comprises between different types or subtypes of a disease or condition such as, for example, different types of pneumonia including viral pneumonia, bacterial pneumonia, mycoplasma pneumonia, fungal pneumonia, idiopathic interstitial pneumonia, or unclassified pneumonia. In some embodiments, the detection or diagnosis comprises a severity and/or stage of a disease or condition such as, for example, different stages of pneumonia (e.g.,

In some embodiments, the predictions or diagnoses generated according to the systems, methods, and devices described herein include detection or diagnosis of a lung disease, disorder, or condition. In some embodiments, the predictions or diagnoses include evaluation of risk or likelihood of pneumonia. In some embodiments, the predictions or diagnosis comprise a category or classification of a type of pneumonia such as bacterial pneumonia, viral pneumonia, fungal pneumonia, mycoplasma pneumonia, or unidentified pneumonia. In some embodiments, the predictions or diagnoses include evaluation of risk or likelihood of childhood pneumonia. In some embodiments, the predictions or diagnoses include evaluation of risk or likelihood of lung diseases or disorders such as emphysema, lung cancer, pneumonia, or tuberculosis. In some embodiments, the predictions or diagnoses include evaluation of risk or likelihood of a heart disease or disorder such as heart failure.

Disclosed herein, in various aspects, are methods incorporating machine learning techniques (e.g., deep learning utilizing convolutional neural networks) that demonstrate great diagnostic power using radiological imagery such as X-rays that leverages databases of X-rays including public databases. Conventional approaches in computer vision using deep learning in other medical fields have encountered significant challenges due to the unavailability of large datasets of labeled medical imagery. Disclosed herein are methods that solve these challenges using innovative methods such as the application of transfer learning.

Accordingly, in some embodiments, provided herein is an AI transfer learning framework for the diagnosis of common lung diseases and disorders with a dataset of X-ray images that is capable of achieving highly accurate diagnosis comparable to human expert performance. In some embodiments, this AI framework categorizes images obtained from pediatric subjects (e.g., children no older than 1, 2, 3, 4, 5, 6, 7, 8, 9, 10, 11, 12, 13, 14, 15, 16, or 17 years old). In some embodiments, normal images are labeled for “observation.” Thus, certain embodiments of the present disclosure utilize the AI framework as a triage system to generate a referral, mimicking real-world applications in community settings, primary care, and urgent care clinics. These embodiments may ultimately confer broad public health impact by promoting earlier diagnosis and detection of disease progression, thereby facilitating treatment that can improve outcomes and quality of life.

In certain aspects, disclosed herein are machine learning frameworks for generating models or classifiers that diagnose one or more lung disorders or conditions. These models or classifiers can be implemented in any of the systems or devices disclosed herein such as diagnostic kiosks or portable devices such as smartphones. As used herein, diagnosing or a diagnosis of a lung disorder or condition can include a prediction or diagnosis of an outcome following a medical procedure. In some embodiments, the machine learning frameworks generate models or classifiers that generate predictions such as, for example, post-operative visual outcomes (e.g., cataract surgery). In some embodiments, the prediction comprises an indication of a lung disease or condition such as, for example, pneumonia.

In some embodiments, the classifier exhibits performance metrics such as accuracy, sensitivity, specificity, positive predictive value, negative predictive value, and/or AUC for an independent sample set. In some embodiments, the classifier exhibits performance metrics such as higher accuracy, sensitivity, specificity, positive predictive value, negative predictive value, and/or AUC for an independent sample set compared to an average human clinician (e.g., an average radiologist). In some embodiments, the classifier provides an accuracy of at least 70%, at least 75%, at least 80%, at least 85%, at least 90%, at least 91%, at least 92%, at least 93%, at least 94%, at least 95%, at least 96%, at least 97%, at least 98%, or at least 99% when tested against at least 100, 200, 300, 400, 500, 600, 700, 800, 900, or 1000 independent samples (e.g., images). In some embodiments, the classifier provides a sensitivity (true positive rate) of at least 70%, at least 75%, at least 80%, at least 85%, at least 90%, at least 91%, at least 92%, at least 93%, at least 94%, at least 95%, at least 96%, at least 97%, at least 98%, at least 99%, and/or a specificity (true negative rate) of at least 70%, at least 75%, at least 80%, at least 85%, at least 90%, at least 91%, at least 92%, at least 93%, at least 94%, at least 95%, at least 96%, at least 97%, at least 98%, or at least 99% when tested against at least 100, 200, 300, 400, 500, 600, 700, 800, 900, or 1000 independent samples (e.g., images). In some embodiments, the classifier provides a positive predictive value (PPV) of at least 70%, at least 75%, at least 80%, at least 85%, at least 90%, at least 91%, at least 92%, at least 93%, at least 94%, at least 95%, at least 96%, at least 97%, at least 98%, or at least 99% when tested against at least 100, 200, 300, 400, 500, 600, 700, 800, 900, or 1000 independent samples (e.g., images). In some embodiments, the classifier provides a negative predictive value (NPV) of at least 70%, at least 75%, at least 80%, at least 85%, at least 90%, at least 91%, at least 92%, at least 93%, at least 94%, at least 95%, at least 96%, at least 97%, at least 98%, or at least 99% when tested against at least 100, 200, 300, 400, 500, 600, 700, 800, 900, or 1000 independent samples (e.g., images). In some embodiments, the classifier has an AUC of at least 0.7, 0.75, 0.8, 0.85, 0.9, 0.91, 0.92, 0.93, 0.94, 0.95, 0.96, 0.97, 0.98 or 0.99 when tested against at least 100, 200, 300, 400, 500, 600, 700, 800, 900, or 1000 independent samples. In some embodiments, the classifier has a weighted error compared to one or more independent experts of no more than 20%, no more than 15%, no more than 12%, no more than 10%, no more than 9%, no more than 8%, no more than 7%, no more than 6%, no more than 5%, no more than 4%, no more than 3%, no more than 2%, or no more than 1% when tested against at least 100, 200, 300, 400, 500, 600, 700, 800, 900, or 1000 independent samples.

Embodiments of the framework disclosed herein demonstrate competitive performance on X-ray modalities without the need for a highly specialized deep learning machine and without a database of millions of example images. Since the distinguishing features of disease are generally more straightforward in X-ray images, the model can perform as well as or better than human experts in diagnosis of X-ray images. Moreover, although the more subtle indicators of pathology and greater variability between images belonging to the same class in X-ray images can negatively impact model accuracy, models generated according to the present framework perform competitively and would still scale in performance with added input images.

According to one aspect of the present disclosure, an occlusion test to identify the areas of greatest importance used by the model in assigning diagnosis is performed. The greatest benefit of an occlusion test is that it reveals insights into the decisions of neural networks, which are sometimes referred to as “black boxes” with no transparency. Since this test is performed after training is completed, it demystifies the algorithm without affecting its results. The occlusion test also confirms that the network makes its decisions using accurate distinguishing features. In some embodiments, various platforms, systems, media, and methods recited herein comprise providing one or more of the areas of greatest importance identified by the occlusion test to a user or subject. In some embodiments, the one or more areas are provided in the form of a report (analog or electronic/digital). In some embodiments, the report is provided to a clinician, the subject of the report, a third party, or a combination thereof. In some embodiments, the report is annotated with medical insight such as descriptions or explanations of how the one or more areas are relevant to the diagnosis. This has the benefit of instilling greater trust and confidence in the methodology. In some embodiments, the medical insight is simplified into layman's terms for a non-clinician or medical practitioner such as the subject or a third party (e.g., parent of the subject). In some embodiments, the report comprises an occlusion image (e.g., image showing areas of greatest importance) used in the diagnosis or prediction. In some embodiments, the machine learning algorithm comprises a neural network. In some embodiments, the neural network comprises at least 1, at least 2, at least 3, at least 4, at least 5, at least 6, at least 7, at least 8, at least 9, at least 10, at least 15, at least 20, at least 25, at least 30, at least 40, at least 50, at least 60, at least 70, at least 80, at least 90, at least 100, at least 150, at least 200, at least 250, at least 300, at least 350, at least 400, at least 450, at least 500, at least 600, at least 700, at least 800, at least 900, at least 1000, at least 5000, or at least 10000 or more neurons or nodes and/or no more than 2, no more than 3, no more than 4, no more than 5, no more than 6, no more than 7, no more than 8, no more than 9, no more than 10, no more than 15, no more than 20, no more than 25, no more than 30, no more than 40, no more than 50, no more than 60, no more than 70, no more than 80, no more than 90, no more than 100, no more than 150, no more than 200, no more than 250, no more than 300, no more than 350, no more than 400, no more than 450, no more than 500, no more than 600, no more than 700, no more than 800, no more than 900, no more than 1000, no more than 5000, or no more than 10000 neurons or nodes. In some embodiments, the number of neurons is limited to below a threshold number in order to prevent overfitting. In some embodiments, the number of neurons is no more than 5, 6, 7, 8, 9, or 10 neurons.

Although transfer learning allows the training of a highly accurate model with a relatively small training dataset, its performance would be inferior to that of a model trained from a random initialization on an extremely large dataset of X-ray images, since even the internal weights can be directly optimized for X-ray feature detection. However, transfer learning using a pre-trained model trained on millions of various medical images can generate a more accurate model when retraining layers for other medical classifications.

The performance of a model can depend highly on the weights of the pre-trained model. Therefore, in some embodiments, the performance of this model is enhanced when tested on a larger ImageNet dataset with more advanced deep learning techniques and architecture described herein. Further, in certain embodiments, the performance of this approach is improved by incorporating ongoing developments in the field of convolutional neural networks applied outside of medical imaging.

In some embodiments, X-ray imaging is used as a demonstration of a generalized approach in medical image interpretation and subsequent decision making. In some embodiments, the subject matter disclosed herein extends the application of artificial intelligence beyond diagnosis or classification of images and into the realm of making treatment recommendations. In some embodiments, the systems, methods, and devices disclosed herein provide one or more treatment recommendations in addition to a diagnosis or detection of a disease or condition such as a lung infection (e.g., pneumonia). In some embodiments, the treatment recommendation further comprises one or more healthcare providers suitable for providing the recommended treatment. In some embodiments, the one or more healthcare providers are selected based on location proximity to the location of the user and/or the system or device providing the recommendation. In some embodiments, the healthcare providers are selected based on available resources for providing the recommended treatment. In some embodiments, additional information for the healthcare providers is provided, which can include estimated time to arrival (for traveling to the provider location), estimated wait time, estimated cost, and/or other information associated with the healthcare providers. In some embodiments, the patient is administered a treatment based on a diagnosed or detected disease or condition. In some embodiments, the patient is administered a recommended treatment based on a diagnosed or detected disease or condition. In some embodiments, the systems, methods, and devices disclosed herein provide a recommendation for further testing. In some embodiments, the further testing comprises blood test, sputum culture, pulse oximetry, chest CT scan, bronchoscopy, pleural fluid culture, tumor biopsy, genetic testing, or other relevant testing to confirm a predicted diagnosis or evaluation. In some embodiments, the systems, methods, and devices disclosed herein provide a recommendation for treatment based on the diagnosis. In some embodiments, a report is generated comprising the diagnosis and any additional relevant information such as, for example, treatment recommendation(s) and prognosis, nearby healthcare providers, or explanation of the diagnosis (optionally customized/personalized depending on the user, e.g., a simple explanation for a patient or a detailed scientific explanation for a clinician). In some embodiments, the healthcare providers are filtered and/or sorted to identify the closest healthcare providers determined to be capable of providing treatment based on the patient's diagnosis. Geographic proximity can be determined based on a threshold cut-off distance between the user or patient (e.g., home address or GPS location from the user's smartphone) and the healthcare provider location. Alternatively, the cut-off can be based on estimated travel time. In some embodiments, the treatment or treatment recommendation is determined based on the diagnosis. As an example, antibiotics may be administered based on a diagnosis of bacterial pneumonia. As another example, anti-viral medication may be administered based on a diagnosis of viral pneumonia.

Various algorithms can be used to generate models that generate a prediction based on the image analysis. In some instances, machine learning methods are applied to the generation of such models (e.g., trained classifier). In some embodiments, the model is generated by providing a machine learning algorithm with training data in which the expected output is known in advance.

In some embodiments, the systems, devices, and methods described herein generate one or more recommendations such as treatment and/or healthcare options for a subject. In some embodiments, the systems, devices, and methods herein comprise a software module providing one or more recommendations to a user. In some embodiments, the treatment and/or healthcare option are specific to the diagnosed disease or condition. For example, a recommendation can suggest a nearby hospital, doctor, or clinic with the requisite facilities or resources for treating the disease or disorder

In some embodiments, a classifier or trained machine learning algorithm of the present disclosure comprises a feature space. In some cases, the classifier comprises two or more feature spaces. The two or more feature spaces may be distinct from one another. In some embodiments, a feature space comprises information such as pixel data from an image. When training the machine learning algorithm, training data such as image data is input into the algorithm which processes the input features to generate a model. In some embodiments, the machine learning algorithm is provided with training data that includes the classification (e.g., diagnostic or test result), thus enabling the algorithm to train by comparing its output with the actual output to modify and improve the model. This is often referred to as supervised learning. Alternatively, in some embodiments, the machine learning algorithm can be provided with unlabeled or unclassified data, which leaves the algorithm to identify hidden structure amongst the cases (referred to as unsupervised learning). Sometimes, unsupervised learning is useful for identifying the features that are most useful for classifying raw data into separate cohorts.

In some embodiments, one or more sets of training data are used to train a machine learning algorithm. Although exemplar embodiments of the present disclosure include machine learning algorithms that use convolutional neural networks, various types of algorithms are contemplated. In some embodiments, the algorithm utilizes a predictive model such as a neural network, a decision tree, a support vector machine, or other applicable model. In some embodiments, the machine learning algorithm is selected from the group consisting of a supervised, semi-supervised and unsupervised learning, such as, for example, a support vector machine (SVM), a Naïve Bayes classification, a random forest, an artificial neural network, a decision tree, a K-means, learning vector quantization (LVQ), self-organizing map (SOM), graphical model, regression algorithm (e.g., linear, logistic, multivariate, association rule learning, deep learning, dimensionality reduction and ensemble selection algorithms. In some embodiments, the machine learning algorithm is selected from the group consisting of: a support vector machine (SVM), a Naïve Bayes classification, a random forest, and an artificial neural network. Machine learning techniques include bagging procedures, boosting procedures, random forest algorithms, and combinations thereof. Illustrative algorithms for analyzing the data include but are not limited to methods that handle large numbers of variables directly such as statistical methods and methods based on machine learning techniques. Statistical methods include penalized logistic regression, prediction analysis of microarrays (PAM), methods based on shrunken centroids, support vector machine analysis, and regularized linear discriminant analysis.

### Diagnostic Platforms, Systems, Devices, and Media

Provided herein, in certain aspects, are platforms, systems, devices, and media for analyzing medical data according to any of the methods of the present disclosure. In some embodiments, the systems and electronic devices are integrated with a program including instructions executable by a processor to carry out analysis of medical data. In some embodiments, the analysis comprises processing at least one medical image with a classifier such as a neural network, optionally trained on non-domain medical images (e.g., medical images not specifically labeled with the desired type of diagnosis) using transfer learning. In some embodiments, the analysis is performed locally on the device utilizing local software integrated into the device. In some embodiments, the analysis is performed remotely on a remote system or server. In some embodiments, the analysis is performed remotely on the cloud after the image is uploaded by the system or device over a network. In some embodiments, the system or device is an existing system or device adapted to interface with a web application operating on the network or cloud for uploading and analyzing image data such as X-ray images. In some embodiments, the system or device provides for portable image storage such as on a USB drive or other portable hard drive. Portable storage enables the images to be transferred to a device capable of performing analysis on the images and/or which has network connectivity for uploading the images for remote analysis on the cloud.

### Cloud-Based Diagnosis

Provided herein, in certain embodiments, are systems, devices, and methods for providing a web application or portal for remote data analysis or diagnosis (e.g., “cloud” diagnosis). In order to tackle the reproducibility and transparency issues brought on by training and testing on a protected or proprietary dataset, such as medical X-ray imagery, provided herein is an easy-to-use application (e.g., web tool) that allows testing of a model on any provided X-ray image. In some embodiments, the application allows a user to load a trained model and predicts the diagnosis of any user-provided image. In some embodiments, the application provides a breakdown of the diagnosis such as generated using softmax probabilities. In some embodiments, the application allows a user to test the algorithm and even upload smartphone captures of X-ray images and yields comparable accuracy. In some embodiments, the application is in communication with a diagnostic or imaging device as described herein. For example, a diagnostic or imaging device used at the point of care such as at a hospital or outside of the clinic setting (e.g., using a portable diagnostic or imaging device at home) can be used to obtain an image of a subject that is then uploaded over a network such as the Internet for remote diagnosis using the application. The diagnosis can then be provided to the user who uploaded the image and/or the subject from whom the image was obtained. In some embodiments, the diagnosis and/or any additional information (e.g., statistical breakdown, instructions, treatment recommendations, etc) is provided to the user and/or subject using e-mail, text messaging, a web portal, regular mail, or other available communication method. In some embodiments, the diagnosis and/or additional information is provided through a secure HIPAA-compliant application or portal (e.g., requiring secured and encrypted login). In some embodiments, the user and/or subject is sent a non-identifying message containing a link and/or information allowing the user or subject to retrieve the diagnosis and/or additional information from a secure storage location such as through a HIPAA-compliant portal.

Furthermore, the disclosed network represents a generalized platform which can potentially apply to a very wide range of medical imaging techniques (e.g., MRI, CT, etc.) to make a clinical diagnostic decision. This could facilitate screening programs and create more efficient referral systems, particularly in remote or low-resource areas, leading to a broad clinical and public health impact.

In some aspects, disclosed herein is a computer-implemented system configured to carry out cloud-based analysis of medical data such as X-ray images. In some embodiments, the system comprises one or more servers operatively coupled to a network. In some embodiments, the system is configured to provide a web portal, including a browser-based web portal, web-based application, or web-based application programming interface (API) accessible by end users on the network. In some embodiments, the web portal comprises an interface for receiving user instructions and/or medical data uploads. In some embodiments, the system receives at least one X-ray image from an end user or electronic device of an end user. In some embodiments, the X-ray image is captured by the electronic device of the end user at the point of care and uploaded to the system on the cloud for analysis. In some embodiments, the web portal is secured by encrypted pass-word protected login. In some embodiments, the system receives uploaded instructions and/or medical data and performs analysis of the medical data using any of the diagnostic methods described herein. In some embodiments, the system generates output from the analysis of the medical data. In some embodiments, the system provides the output of the analysis to the end user on the network. In some embodiments, the system sends the output to an electronic device of the end user such as a computer, smartphone, tablet or other digital processing device configured for network communications.

### Hardware/Software Integration

Disclosed herein, in some aspects, are electronic devices comprising software configured for performing the machine learning algorithms described herein. In some embodiments, the electronic device comprises an imaging component for capturing an image of a subject, a user interface for communicating with and/or receiving instructions from a user or subject, a memory, at least one processor, and non-transitory computer readable media providing instructions executable by the at least one processor for performing analysis of the captured image. In some embodiments, the electronic device comprises a network component for communicating with a network or cloud. The network component is configured to communicate over a network using wired or wireless technology. In some embodiments, the network component communicates over a network using Wi-Fi, Bluetooth, 2G, 3G, 4G, 4G LTE, 5G, WiMAX, WiMAN, or other radiofrequency communication standards and protocols.

In some embodiments, the system or electronic device captures a plurality of images for analysis. In some embodiments, the plurality of images are merged and/or analyzed collectively. In some embodiments, the electronic device is not configured to carry out analysis of the captured image locally, instead uploading the captured image to a network for cloud-based or remote analysis. In some embodiments, the electronic device comprises a web portal application that interfaces with the network or cloud for remote analysis and does not carry out any analysis locally. An advantage of this configuration is that image data is not stored locally and thus less vulnerable to being hacked or lost. Alternatively or in combination, the electronic device is configured to carry out analysis of the captured image locally. An advantage of this configuration is the ability to perform analysis in locations lacking network access or coverage (e.g., in certain remote locations lacking internet coverage). In some embodiments, the electronic device is configured to carry out analysis of the captured image locally when network access is not available as a backup function such as in case of an internet outage or temporary network failure. In some embodiments, the image data is uploaded for storage on the cloud regardless of where the analysis is carried out. For example, in certain instances, the image data is temporarily stored on the electronic device for analysis, and subsequently uploaded on the cloud and/or deleted from the electronic device's local memory.

In some embodiments, the system comprises the electronic device and cloud-based server(s) carrying out the analysis and/or storing the image data. In some embodiments, the system comprises the electronic device and an imaging component physically separate from the electronic device. As an example, the system comprises an electronic device that is a desktop computer coupled to or otherwise in communication with an imaging component (e.g., X-ray). In some embodiments, the system allows for an image to be captured using the imaging component, and the analysis to be performed by the electronic device, or alternatively, by the cloud following upload of the image. In some embodiments, the system comprises the electronic device for analyzing and/or uploading an image, an imaging component for capturing an image and configured to send the image or image data to the electronic device, and a cloud-based server for receiving an uploaded image and storing and/or analyzing the image, and generating a result to be provided to a user via the electronic device or other methods such as by messaging, email, or a phone call. In some embodiments, the system or device comprises a plurality of imaging components. In some embodiments, the plurality of imaging components is configured to capture multiple types of images. In some embodiments, analysis of the multiple types of images is carried out by different classifiers trained on the different image types to provide more than one diagnosis or result. Alternatively, in some embodiments, the more than one diagnosis or result is consolidated or combined into a single result metric (e.g., an average of the predictions for a particular disorder such as viral or bacterial pneumonia).

In some embodiments, the electronic device comprises a display for providing the results of the analysis such as a diagnosis or prediction (of the presence and/or progression of a disease or disorder), a treatment recommendation, treatment options, healthcare provider information (e.g., nearby providers that can provide the recommended treatment and/or confirm the diagnosis), or a combination thereof. In some embodiments, the diagnosis or prediction is generated from analysis of the captured image in comparison to previously captured image(s) for the same user to determine the progression of a disease or disorder. In some embodiments, captured images are time-stamped. In some embodiments, captured images are stored as data, which optionally includes meta-data such as a timestamp, location, user info, or other information associated with the images). In some embodiments, the image data is screened for quality. In some embodiments, the image is screened for suitability for analysis. In some embodiments, an image failing the screen is discarded or otherwise rejected from further analysis. In some embodiments, the electronic device prompts a user to take one or more additional images.

In some embodiments, the electronic device comprises a portal providing one or more tools for a user to input information such as name, address, email, phone number, and/or other identifying information. In some embodiments, the portal comprises an interface for obtaining or entering medical data. In some embodiments, the portal is configured to receive medical data for use in the prediction or diagnosis from device through a network (e.g., receives medical data provided by a user smartphone through the internet via a mobile app or web portal). In some embodiments, the medical data comprises medical information such as diagnosis, treatment recommendations, medical history, or recommended healthcare providers (e.g., providers capable of treating the diagnosed disease or disorder within a certain geographic proximity of the user location).

In some embodiments, the portal is configured to provide a health assessment through the electronic device. In some embodiments, the health assessment comprises a diagnosis of a disease or condition. In some embodiments, the disease or condition is a lung disease or condition. In some embodiments, the lung disease or condition is pneumonia. In some embodiments, the pneumonia is viral pneumonia, bacterial pneumonia, mycoplasma pneumonia, or fungal pneumonia.

In some embodiments, the portal provides the user with the option to receive the results of the analysis by email, messaging (e.g., SMS, text message), physical printout (e.g., a printed report), social media, by phone (e.g., an automated phone message or a consultation by a healthcare provider or adviser), or a combination thereof. In some embodiments, the captured image(s) is provided to the user. For example, an image can be shown with graphical emphasis (e.g., highlighting, boundaries drawn around the areas, zoomed in view, etc) on the areas that are most important to the diagnosis as identified by the occlusion test, which can help promote understanding and trust in the diagnostic method. In some embodiments, the portal is displayed on a digital screen of the electronic device. In some embodiments, the electronic device comprises an analog interface. In some embodiments, the electronic device comprises a digital interface such as a touchscreen. In various embodiments, existing systems and devices are capable of being adapted to carry out the methods disclosed herein or are capable of interfacing with web applications for performing remote analysis of X-ray images.

In some embodiments, the electronic device has a hardware configuration adapted for capturing images of a subject for analysis according to the methods described herein. In some embodiments, the electronic device comprises a specialized imaging component such as an X-ray machine. In some embodiments, the computer and X-ray machine configured as a single integrated unit.

### Digital Processing Device

In some embodiments, the systems, devices, platforms, media, methods and applications described herein include a digital processing device, a processor, or use of the same. For example, in some embodiments, the digital processing device is part of a point-of-care device such as a medical diagnostic device integrating the diagnostic software described herein. In some embodiments, the medical diagnostic device is a consumer-facing portable medical diagnostic device configured for use outside of the clinical setting (e.g., consumer use at home). For example, a consumer may utilize a smartphone configured with the diagnostic software described herein to capture a medical image (e.g., take a picture of an X-ray) for analysis. The smartphone may perform the diagnosis or evaluation of the medical image locally or upload it to the cloud for remote analysis. In some embodiments, the medical diagnostic device comprises diagnostic equipment such as imaging hardware (e.g., a X-ray machine) for capturing medical data (e.g., X-ray images). In some embodiments, the medical diagnostic device comprises a digital processing device configured to perform the diagnostic methods described herein such as disease detection or classification based on medical images. In further embodiments, the digital processing device includes one or more processors or hardware central processing units (CPU) that carry out the device's functions. In still further embodiments, the digital processing device further comprises an operating system configured to perform executable instructions. In some embodiments, the digital processing device is optionally connected a computer network. In further embodiments, the digital processing device is optionally connected to the Internet such that it accesses the World Wide Web. In still further embodiments, the digital processing device is optionally connected to a cloud computing infrastructure. In other embodiments, the digital processing device is optionally connected to an intranet. In other embodiments, the digital processing device is optionally connected to a data storage device. In accordance with the description herein, suitable digital processing devices include, by way of non-limiting examples, server computers, desktop computers, laptop computers, notebook computers, sub-notebook computers, netbook computers, netpad computers, set-top computers, handheld computers, Internet appliances, mobile smartphones, tablet computers, personal digital assistants, video game consoles, and vehicles. Those of skill in the art will recognize that many smartphones are suitable for use in the system described herein. Those of skill in the art will also recognize that select televisions, video players, and digital music players with optional computer network connectivity are suitable for use in the system described herein. Suitable tablet computers include those with booklet, slate, and convertible configurations, known to those of skill in the art.

In some embodiments, the digital processing device includes an operating system configured to perform executable instructions. The operating system is, for example, software, including programs and data, which manages the device's hardware and provides services for execution of applications. Those of skill in the art will recognize that suitable server operating systems include, by way of non-limiting examples, FreeBSD, OpenBSD, NetBSD®, Linux, Apple® Mac OS X Server®, Oracle® Solaris®, Windows Server®, and Novell® NetWare®. Those of skill in the art will recognize that suitable personal computer operating systems include, by way of non-limiting examples, Microsoft® Windows®, Apple® Mac OS X®, UNIX®, and UNIX-like operating systems such as GNU/Linux®. In some embodiments, the operating system is provided by cloud computing. Those of skill in the art will also recognize that suitable mobile smart phone operating systems include, by way of non-limiting examples, Nokia® Symbian® OS, Apple® iOS®, Research In Motion® BlackBerry OS®, Google Android®, Microsoft® Windows Phone® OS, Microsoft® Windows Mobile® OS, Linux®, and Palm® WebOS®.

In some embodiments, the device includes a storage and/or memory device. The storage and/or memory device is one or more physical apparatuses used to store data or programs on a temporary or permanent basis. In some embodiments, the device is volatile memory and requires power to maintain stored information. In some embodiments, the device is non-volatile memory and retains stored information when the digital processing device is not powered. In further embodiments, the non-volatile memory comprises flash memory. In some embodiments, the non-volatile memory comprises dynamic random-access memory (DRAM). In some embodiments, the non-volatile memory comprises ferroelectric random access memory (FRAM). In some embodiments, the non-volatile memory comprises phase-change random access memory (PRAM). In some embodiments, the non-volatile memory comprises magnetoresistive random-access memory (MRAM). In other embodiments, the device is a storage device including, by way of non-limiting examples, CD-ROMs, DVDs, flash memory devices, magnetic disk drives, magnetic tapes drives, optical disk drives, and cloud computing based storage. In further embodiments, the storage and/or memory device is a combination of devices such as those disclosed herein.

In some embodiments, the digital processing device includes a display to send visual information to a subject. In some embodiments, the display is a cathode ray tube (CRT). In some embodiments, the display is a liquid crystal display (LCD). In further embodiments, the display is a thin film transistor liquid crystal display (TFT-LCD). In some embodiments, the display is an organic light emitting diode (OLED) display. In various further embodiments, on OLED display is a passive-matrix OLED (PMOLED) or active-matrix OLED (AMOLED) display. In some embodiments, the display is a plasma display. In some embodiments, the display is E-paper or E ink. In other embodiments, the display is a video projector. In still further embodiments, the display is a combination of devices such as those disclosed herein.

In some embodiments, the digital processing device includes an input device to receive information from a subject. In some embodiments, the input device is a keyboard. In some embodiments, the input device is a pointing device including, by way of non-limiting examples, a mouse, trackball, track pad, joystick, game controller, or stylus. In some embodiments, the input device is a touch screen or a multi-touch screen. In other embodiments, the input device is a microphone to capture voice or other sound input. In other embodiments, the input device is a video camera or other sensor to capture motion or visual input. In further embodiments, the input device is a Kinect, Leap Motion, or the like. In still further embodiments, the input device is a combination of devices such as those disclosed herein.

### Non-Transitory Computer Readable Storage Medium

In some embodiments, the platforms, media, methods and applications described herein include one or more non-transitory computer readable storage media encoded with a program including instructions executable by the operating system of an optionally networked digital processing device. In further embodiments, a computer readable storage medium is a tangible component of a digital processing device. In still further embodiments, a computer readable storage medium is optionally removable from a digital processing device. In some embodiments, a computer readable storage medium includes, by way of non-limiting examples, CD-ROMs, DVDs, flash memory devices, solid state memory, magnetic disk drives, magnetic tape drives, optical disk drives, cloud computing systems and services, and the like. In some cases, the program and instructions are permanently, substantially permanently, semi-permanently, or non-transitorily encoded on the media.

### Computer Program

In some embodiments, the platforms, media, methods and applications described herein include at least one computer program, or use of the same. A computer program includes a sequence of instructions, executable in the digital processing device's CPU, written to perform a specified task. Computer readable instructions may be implemented as program modules, such as functions, objects, Application Programming Interfaces (APIs), data structures, and the like, that perform particular tasks or implement particular abstract data types. In light of the disclosure provided herein, those of skill in the art will recognize that a computer program may be written in various versions of various languages.

The functionality of the computer readable instructions may be combined or distributed as desired in various environments. In some embodiments, a computer program comprises one sequence of instructions. In some embodiments, a computer program comprises a plurality of sequences of instructions. In some embodiments, a computer program is provided from one location. In other embodiments, a computer program is provided from a plurality of locations. In various embodiments, a computer program includes one or more software modules. In various embodiments, a computer program includes, in part or in whole, one or more web applications, one or more mobile applications, one or more standalone applications, one or more web browser plug-ins, extensions, add-ins, or add-ons, or combinations thereof.

### Web Application

In some embodiments, a computer program includes a web application. In light of the disclosure provided herein, those of skill in the art will recognize that a web application, in various embodiments, utilizes one or more software frameworks and one or more database systems. In some embodiments, a web application is created upon a software framework such as Microsoft® .NET or Ruby on Rails (RoR). In some embodiments, a web application utilizes one or more database systems including, by way of non-limiting examples, relational, non-relational, object oriented, associative, and XML database systems. In further embodiments, suitable relational database systems include, by way of non-limiting examples, Microsoft® SQL Server, mySQL™, and Oracle®. Those of skill in the art will also recognize that a web application, in various embodiments, is written in one or more versions of one or more languages. A web application may be written in one or more markup languages, presentation definition languages, client-side scripting languages, server-side coding languages, database query languages, or combinations thereof. In some embodiments, a web application is written to some extent in a markup language such as Hypertext Markup Language (HTML), Extensible Hypertext Markup Language (XHTML), or eXtensible Markup Language (XML). In some embodiments, a web application is written to some extent in a presentation definition language such as Cascading Style Sheets (CSS). In some embodiments, a web application is written to some extent in a client-side scripting language such as Asynchronous Javascript and XML (AJAX), Flash® Actionscript, Javascript, or Silverlight®. In some embodiments, a web application is written to some extent in a server-side coding language such as Active Server Pages (ASP), ColdFusion®, Perl, Java™, JavaServer Pages (JSP), Hypertext Preprocessor (PHP), Python™, Ruby, Tcl, Smalltalk, WebDNA®, or Groovy. In some embodiments, a web application is written to some extent in a database query language such as Structured Query Language (SQL). In some embodiments, a web application integrates enterprise server products such as IBM® Lotus Domino®. In some embodiments, a web application includes a media player element. In various further embodiments, a media player element utilizes one or more of many suitable multimedia technologies including, by way of non-limiting examples, Adobe® Flash®, HTML 5, Apple® QuickTime®, Microsoft® Silverlight®, Java™, and Unity®.

### Mobile Application

In some embodiments, a computer program includes a mobile application provided to a mobile digital processing device such as a smartphone. In some embodiments, the mobile application is provided to a mobile digital processing device at the time it is manufactured. In other embodiments, the mobile application is provided to a mobile digital processing device via the computer network described herein.

In view of the disclosure provided herein, a mobile application is created by techniques known to those of skill in the art using hardware, languages, and development environments known to the art. Those of skill in the art will recognize that mobile applications are written in several languages. Suitable programming languages include, by way of non-limiting examples, C, C++, C#, Objective-C, Java™, Javascript, Pascal, Object Pascal, Python™, Ruby, VB.NET, WML, and XHTML/HTML with or without CSS, or combinations thereof.

Suitable mobile application development environments are available from several sources. Commercially available development environments include, by way of non-limiting examples, AirplaySDK, alcheMo, Appcelerator®, Celsius, Bedrock, Flash Lite, .NET Compact Framework, Rhomobile, and WorkLight Mobile Platform. Other development environments are available without cost including, by way of non-limiting examples, Lazarus, MobiFlex, MoSync, and Phonegap. Also, mobile device manufacturers distribute software developer kits including, by way of non-limiting examples, iPhone and iPad (iOS) SDK, Android™ SDK, BlackBerry® SDK, BREW SDK, Palm® OS SDK, Symbian SDK, webOS SDK, and Windows® Mobile SDK.

Those of skill in the art will recognize that several commercial forums are available for distribution of mobile applications including, by way of non-limiting examples, Apple® App Store, Android™ Market, BlackBerry® App World, App Store for Palm devices, App Catalog for webOS, Windows® Marketplace for Mobile, Ovi Store for Nokia® devices, Samsung® Apps, and Nintendo® DSi Shop.

### Standalone Application

In some embodiments, a computer program includes a standalone application, which is a program that is run as an independent computer process, not an add-on to an existing process, e.g., not a plug-in. Those of skill in the art will recognize that standalone applications are often compiled. A compiler is a computer program(s) that transforms source code written in a programming language into binary object code such as assembly language or machine code. Suitable compiled programming languages include, by way of non-limiting examples, C, C++, Objective-C, COBOL, Delphi, Eiffel, Java™, Lisp, Python™, Visual Basic, and VB .NET, or combinations thereof. Compilation is often performed, at least in part, to create an executable program. In some embodiments, a computer program includes one or more executable complied applications.

### Software Modules

In some embodiments, the platforms, media, methods and applications described herein include software, server, and/or database modules, or use of the same. In view of the disclosure provided herein, software modules are created by techniques known to those of skill in the art using machines, software, and languages known to the art. The software modules disclosed herein are implemented in a multitude of ways. In various embodiments, a software module comprises a file, a section of code, a programming object, a programming structure, or combinations thereof. In further various embodiments, a software module comprises a plurality of files, a plurality of sections of code, a plurality of programming objects, a plurality of programming structures, or combinations thereof. In various embodiments, the one or more software modules comprise, by way of non-limiting examples, a web application, a mobile application, and a standalone application. In some embodiments, software modules are in one computer program or application. In other embodiments, software modules are in more than one computer program or application. In some embodiments, software modules are hosted on one machine. In other embodiments, software modules are hosted on more than one machine. In further embodiments, software modules are hosted on cloud computing platforms. In some embodiments, software modules are hosted on one or more machines in one location. In other embodiments, software modules are hosted on one or more machines in more than one location.

### Databases

In some embodiments, the platforms, systems, media, and methods disclosed herein include one or more databases, or use of the same. In view of the disclosure provided herein, those of skill in the art will recognize that many databases are suitable for storage and retrieval of barcode, route, parcel, subject, or network information. In various embodiments, suitable databases include, by way of non-limiting examples, relational databases, non-relational databases, object oriented databases, object databases, entity-relationship model databases, associative databases, and XML databases. In some embodiments, a database is internet-based. In further embodiments, a database is web-based. In still further embodiments, a database is cloud computing-based. In other embodiments, a database is based on one or more local computer storage devices.

### NUMBERED EMBODIMENTS

The disclosure is further elucidated by reference to the numbered embodiments herein. 1. A method for providing a medical diagnosis, comprising: a) obtaining a medical image of a lung; b) performing a machine learning procedure on the medical image of the lung; and c) determining, by the machine learning procedure, whether or not the medical image of the lung is indicative of a disease or disorder of the lung, the determination having a sensitivity greater than 90% and a specificity greater than 90%. 2. The method of embodiment 1, wherein the machine learning procedure comprises a deep learning procedure. 3. The method of embodiment 1 or 2, wherein the machine learning procedure comprises a convolutional neural network. 4. The method of any one of embodiments 1-3, further comprising subjecting the medical image of the lung to an image occlusion procedure. 5. The method of any one of embodiments 1-4, further comprising performing a transfer learning procedure. 6. The method of embodiment 5, wherein the transfer learning procedure comprises pre-training the machine learning procedure using non-medical or unlabeled medical images obtained from a large image dataset to obtain a pre-trained machine learning procedure. 7. The method of embodiment 6, wherein the transfer learning procedure further comprises training the pre-trained machine learning procedure using a set of medical images that is smaller than the large image dataset. 8. The method of any one of embodiments 1-7, further comprising making a medical treatment recommendation based on the determination. 9. The method of any one of embodiments 1-8, wherein the medical image of the lung is a chest X-ray. 10. The method of any one of embodiments 1-9, wherein the medical image comprises an X-ray image. 11. The method of any one of embodiments 1-10, wherein the medical image comprises a plurality of lung X-rays. 12. The method of any one of embodiments 1-11, wherein the disease or disorder of the lung is selected from the group consisting of: pneumonia, childhood pneumonia, emphysema, and lung cancer. 13. A non-transitory computer-readable medium comprising machine-executable code that, upon execution by one or more computer processors, implements a method for providing a medical diagnosis, the method comprising: a) obtaining a medical image of a lung; b) performing a machine learning procedure on the medical image of the lung; and c) determining, by the machine learning procedure, whether or not the medical image of the lung is indicative of a disease or disorder of the lung, the determination having a sensitivity greater than 90% and a specificity greater than 90%. 14. The non-transitory computer-readable medium of embodiment 13, wherein the machine learning procedure comprises a deep learning procedure. 15. The non-transitory computer-readable medium of embodiment 13 or 14, wherein the machine learning procedure comprises a convolutional neural network. 16. The non-transitory computer-readable medium of any one of embodiments 13-15, wherein the method further comprises subjecting the medical image of the lung to an image occlusion procedure. 17. The non-transitory computer-readable medium of any one of embodiments 13-16, wherein the method further comprises performing a transfer learning procedure. 18. The non-transitory computer-readable medium of embodiment 17, wherein the transfer learning procedure comprises pre-training the machine learning procedure using non-medical or unlabeled medical images obtained from a large image dataset to obtain a pre-trained machine learning procedure. 19. The non-transitory computer-readable medium of embodiment 18, wherein the transfer learning procedure further comprises training the pre-trained machine learning procedure using a set of medical images that is smaller than the large image dataset. 20. The non-transitory computer-readable medium of any one of embodiments 13-19, wherein the method further comprises making a medical treatment recommendation based on the determination. 21. The non-transitory computer-readable medium of any one of embodiments 13-20, wherein the medical image of the lung is a chest X-ray. 22. The non-transitory computer-readable medium of any one of embodiments 13-21, wherein the medical image comprises n X-ray image. 23. The non-transitory computer-readable medium of any one of embodiments 13-22, wherein the medical image comprises a plurality of lung X-rays. 24. The non-transitory computer-readable medium of any one of embodiments 13-23, wherein the disease or disorder of the lung is selected from the group consisting of: pneumonia, childhood pneumonia, emphysema, and lung cancer. 25. A computer-implemented system comprising: a digital processing device comprising: at least one processor, an operating system configured to perform executable instructions, a memory, and a computer program including instructions executable by the digital processing device to create an application for providing a medical diagnosis of a disease or disorder or a lung, the application comprising: a) a software module for obtaining a medical image of a lung; b) a software module for performing a machine learning procedure on the medical image of the lung; and c) a software module for determining, by the machine learning procedure, whether or not the medical image of the lung is indicative of a disease or disorder of the lung, the determination having a sensitivity greater than 90% and a specificity greater than 90%. 26. The computer-implemented system of embodiment 25, wherein the machine learning procedure comprises a deep learning procedure. 27. The computer-implemented system of embodiment 25 or 26, wherein the machine learning procedure comprises a convolutional neural network. 28. The computer-implemented system of any one of embodiments 25-27, wherein the application further comprises a software module for subjecting the medical image of the lung to an image occlusion procedure. 29. The computer-implemented system of any one of embodiments 25-28, wherein the application further comprises a software module for performing a transfer learning procedure. 30. The computer-implemented system of embodiment 29, wherein the transfer learning procedure comprises pre-training the machine learning procedure using non-medical or unlabeled medical images obtained from a large image dataset to obtain a pre-trained machine learning procedure. 31. The computer-implemented system of embodiment 30, wherein the transfer learning procedure further comprises training the pre-trained machine learning procedure using a set of medical images that is smaller than the large image dataset. 32. The computer-implemented system of any one of embodiments 25-31, wherein the application further comprises a software module for making a medical treatment recommendation based on the determination. 33. The computer-implemented system of any one of embodiments 25-32, wherein the medical image of the lung is a chest X-ray. 34. The computer-implemented method of any one of embodiments 25-33 wherein the medical image comprises an X-ray image. 35. The computer-implemented method of any one of embodiments 25-34, wherein the medical image comprises a plurality of lung X-rays. 36. The computer-implemented system of any one of embodiments 25-33, wherein the disease or disorder of the lung is selected from the group consisting of: pneumonia, childhood pneumonia, emphysema, and lung cancer. 37. A method for providing a medical diagnosis, comprising: a) obtaining a medical image of a lung; b) evaluating the medical image using a predictive model trained using a machine learning procedure; and c) determining, by the predictive model, whether or not the medical image is indicative of a disease or disorder of the lung, the determination having a sensitivity greater than 90% and a specificity greater than 90%. 38. The method of embodiment 37, wherein the machine learning procedure comprises a deep learning procedure. 39. The method of embodiment 37 or 38, wherein the machine learning procedure comprises a convolutional neural network. 40. The method of any one of embodiments 37-39, further comprising subjecting the medical image of the lung to an image occlusion procedure. 41. The method of any one of embodiments 37-40, wherein the machine learning procedure comprises a transfer learning procedure. 42. The method of embodiment 41, wherein the transfer learning procedure comprises pre-training the machine learning procedure using non-medical or unlabeled medical images obtained from a large image dataset to obtain a pre-trained model. 43. The method of embodiment 42, wherein the transfer learning procedure further comprises training the pre-trained model using a set of medical images that is smaller than the large image dataset. 44. The method of any one of embodiments 37-43, further comprising making a medical treatment recommendation based on the determination. 45. The method of any one of embodiments 37-44, wherein the medical image of the lung is a chest X-ray. 46. The method of any one of embodiments 37-45, wherein the medical image comprises an X-ray image. 47. The method of any one of embodiments 37-46, wherein the medical image comprises a plurality of lung X-rays. 48. The method of any one of embodiments 37-47, wherein the disease or disorder of the lung is selected from the group consisting of: pneumonia, childhood pneumonia, emphysema, tuberculosis, and lung cancer. 49. A non-transitory computer-readable medium comprising machine-executable code that, upon execution by one or more computer processors, implements a method for providing a medical diagnosis, the method comprising: a) obtaining a medical image of a lung; b) evaluating the medical image using a predictive model trained using a machine learning procedure; and c) determining, by the predictive model, whether or not the medical image is indicative of a disease or disorder of the lung, the determination having a sensitivity greater than 90% and a specificity greater than 90%. 50. The non-transitory computer-readable medium of embodiment 49, wherein the machine learning procedure comprises a deep learning procedure. 51. The non-transitory computer-readable medium of embodiment 49 or 50, wherein the machine learning procedure comprises a convolutional neural network. 52. The non-transitory computer-readable medium of any one of embodiments 49-51, wherein the method further comprises subjecting the medical image of the lung to an image occlusion procedure. 53. The non-transitory computer-readable medium of any one of embodiments 49-52, wherein the machine learning procedure comprises a transfer learning procedure. 54. The non-transitory computer-readable medium of embodiment 53, wherein the transfer learning procedure comprises pre-training the machine learning procedure using non-medical or unlabeled medical images obtained from a large image dataset to obtain a pre-trained model. 55. The non-transitory computer-readable medium of embodiment 54, wherein the transfer learning procedure further comprises training the pre-trained model using a set of medical images that is smaller than the large image dataset. 56. The non-transitory computer-readable medium of any one of embodiments 49-55, wherein the method further comprises making a medical treatment recommendation based on the determination. 57. The non-transitory computer-readable medium of any one of embodiments 49-56, wherein the medical image of the lung is a chest X-ray. 58. The non-transitory computer-readable medium of any one of embodiments 49-57, wherein the medical image comprises an X-ray image. 59. The non-transitory computer-readable medium of any one of embodiments 49-58, wherein the medical image comprises a plurality of lung X-rays. 60. The non-transitory computer-readable medium of any one of embodiments 49-59, wherein the disease or disorder of the lung is selected from the group consisting of: pneumonia, childhood pneumonia, emphysema, tuberculosis, and lung cancer. 61. A computer-implemented system comprising: a digital processing device comprising: at least one processor, an operating system configured to perform executable instructions, a memory, and a computer program including instructions executable by the digital processing device to create an application for providing a medical diagnosis of a disease or disorder or a lung, the application comprising: a) a software module for obtaining a medical image of a lung; b) a software module for analyzing the medical image using a predictive model trained using a machine learning procedure; and c) a software module for determining, by the predictive model, whether or not the medical image of the lung is indicative of a disease or disorder of the lung, the determination having a sensitivity greater than 90% and a specificity greater than 90%. 62. The system of embodiment 61, wherein the machine learning procedure comprises a deep learning procedure. 63. The system of embodiment 61 or 62, wherein the machine learning procedure comprises a convolutional neural network. 64. The system of any one of embodiments 61-63, wherein the application further comprises a software module for subjecting the medical image of the lung to an image occlusion procedure. 65. The system of any one of embodiments 61-64, wherein the machine learning procedure comprises a transfer learning procedure. 66. The system of embodiment 65, wherein the transfer learning procedure comprises pre-training the machine learning procedure using non-domain medical images obtained from a large image dataset to obtain a pre-trained model. 67. The system of embodiment 66, wherein the transfer learning procedure further comprises training the pre-trained model using a set of labeled medical images that is smaller than the large image dataset. 68. The system of any one of embodiments 61-67, wherein the application further comprises a software module for making a medical treatment recommendation based on the determination. 69. The system of any one of embodiments 61-68, wherein the medical image of the lung is a chest X-ray. 70. The system of any one of embodiments 61-69, wherein the medical image comprises an X-ray image. 71. The system of any one of embodiments 61-70, wherein the medical image comprises a plurality of lung X-rays. 72. The system of any one of embodiments 61-71, wherein the disease or disorder of the lung is selected from the group consisting of: pneumonia, childhood pneumonia, emphysema, tuberculosis, and lung cancer. 73. The system of any one of embodiments 61-72, further comprising an imaging device in operative communication with the digital processing device. 74. The method, medium, or system of any of the preceding embodiments, wherein the determination is made by uploading the image to a cloud for remote analysis and receiving the determination generated by the cloud. 75. The method, medium, or system of any of the preceding embodiments, wherein the transfer learning procedure comprises pre-training a first model on a first image data set, freezing at least a portion of the first model, generating a second model comprising the at least a portion of the first model, and training the second model on a second image data set labeled with a diagnostic status of the disease or condition.

### EXAMPLES

### Example 1

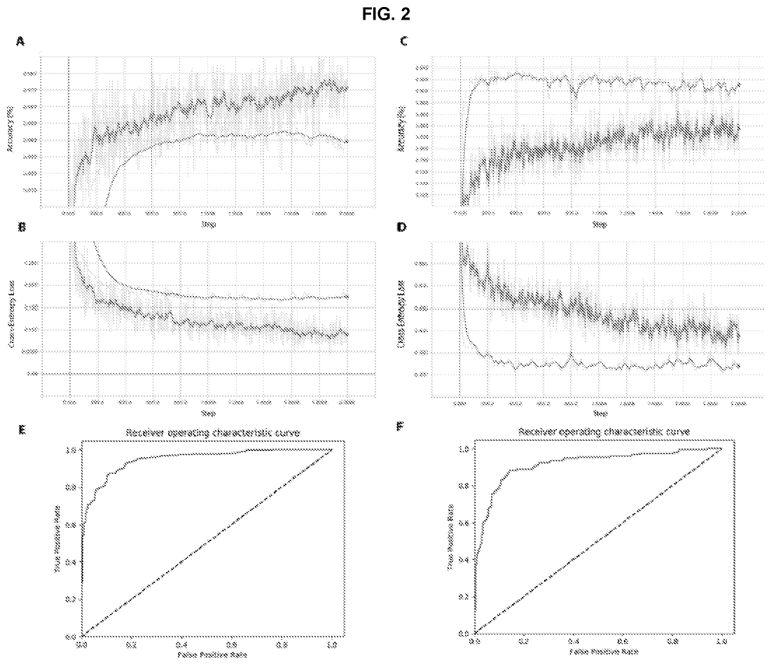

To investigate the generalizability of the AI system in the diagnosis of common diseases, the same transfer learning framework was applied to the diagnosis of pediatric pneumonia. Pneumonia is the leading cause of childhood mortality worldwide. Effective (and often lifesaving) treatment depends on timely and accurate diagnosis, particularly for bacterial pneumonia which necessitates urgent antibiotic treatment. Chest radiographs are often a key component in the diagnosis of pneumonia. A total of 5,232 chest x-ray images from children were collected and labeled, including 3,883 characterized as depicting pneumonia (2,538 bacterial and 1,345 viral) and 1,349 normal from 5,856 patients, to train the AI system. The model was then tested with 234 normal images and 390 pneumonia images (242 bacterial and 148 viral) from 624 patients. After 100 epochs (iterations through the entire dataset) of the model, the training was stopped due to the absence of further improvement in both loss and accuracy (FIGS. 2, A and B). In the comparison of chest x-rays presenting as pneumonia versus normal, the model achieved an accuracy of 94.8%, with a sensitivity of 95.2% and a specificity of 91.9%. An ROC curve with pneumonia as the positive case generated an area under the curve of 98.1% (FIG. 2, E). Binary comparison of bacterial and viral pneumonia resulted in a test accuracy of 93.7%, with a sensitivity of 89.9% and a specificity of 92.3% (FIGS. 2, C and 2D). An ROC curve was generated using bacterial pneumonia as the positive case, with an area under the curve of 95.0% (FIG. 2, F).

Occlusion testing was performed to identify the areas contributing most to the neural network's assignment of the predicted diagnosis. This testing successfully identified the region of interest that contributed the highest importance to the deep learning algorithm. Furthermore, these regions were consistent with what human experts deemed to be clinically significant areas of pathology.

Training the chest X-ray images required grading to determine a ground-truth label for each image, followed by preprocessing involving cropping of images to only include the chest. The images were separated into a training set (460 viral and 460 bacterial) and a validation set (67 viral and 67 bacterial). The PyTorch framework with a GTX 970 GPU was used for training the final layer of a state-of-the-art ResNet-50 architecture pretrained on the ImageNet dataset. During training, the data was artificially augmented using a random cropping of 224×224 pixels and random horizontal flipping in order to strengthen the small dataset and allow the model to minimize overfitting. Using stochastic gradient descent (SGD) as the optimizer during training, an initial learning rate of 0.1 was used with an exponential decrease every 10 epochs. The training was executed over 50 epochs, or iterations through the entire dataset, and validation was performed after each epoch to measure current performance. The model weights with the best validation performance was saved as the best model with an accuracy of 66%. After removing the images previously marked incorrect and rerunning training, the model with the best accuracy reported 78.7% accuracy.

### Methods

### Datasets

### Image Labelling

Before training, each image went through a tiered grading system consisting of multiple layers of trained graders of increasing expertise for verification and correction of image labels. Each image imported into the database started with a label matching the most recent diagnosis of the patient. Scans where no diagnostic label could be attached were also excluded. The presence or absence of pathologies visible on the image was recorded.

### Transfer Learning Methods

A residual DNN called ResNet (He et al, 2016) and a multilayer feedforward DNN Inception (Szegedy et al, 2015) were adapted to carry out the analysis. The pretrained Inception-v3 architecture was implemented in Tensorflow, and the three pretrained ResNet variants (ResNet-50, -101, -152) were implemented in PyTorch. While the ResNet variants have shown significantly less error in the ImageNet dataset, the Inception model yielded slightly higher accuracy.

With both models, retraining consisted of initializing the convolutional layers with the pretrained weights and retraining the final, softmax layer to recognize the classes from scratch. In this study, the convolutional layers were frozen and used as fixed feature extractors. The convolutional “bottlenecks” are the values of each training and testing images after it has passed through the frozen layers of the model and since the convolutional weights are not updated, these values are initially calculated and stored in order to reduce redundant processes and speed up training. Attempts at “fine-tuning” the convolutional layers by training the pretrained weights to the medical images using backpropagation tended to decrease performance by overfitting.

The Inception model was trained on an Ubuntu 16.04 computer with 2 Intel Xeon CPUs, using a NVIDIA GTX 1080 8 Gb GPU for training and testing, with 256 Gb available in RAM memory. Training of layers done by stochastic gradient descent in batches of 1,000 images per step using an Adam Optimizer with a learning rate of 0.001. Training on all categories was run for 10,000 steps, or 100 epochs, since training will have converged by then for all classes. Validation was performed after every step and the best performing model was kept for analysis.

The ResNet variants were trained using an Ubuntu 16.04 computer with an Intel i5-4690k CPU, using a NVIDIA GTX 970 4 Gb GPU for training and testing, with 4 Gb available in RAM memory. Training used stochastic gradient descent in batches of 8 images with an initial learning rate of 0.1 that is exponentially reduced by a factor of 0.1 every 7 epochs. Training on classes was run for 100 epochs. Validation was performed after every epoch and the best performing model was kept for analysis.

### Expert Comparisons

In order to evaluate the model in the context of clinical experts, a validation set of 1000 images (633 patients) was used to compare the network referral decisions with the decisions made by human experts. Weighted error scoring was used to reflect the fact that a false negative result (failing to refer) is more detrimental than a false positive result (making a referral when it was not warranted). Using these weighted penalty points, error rates were computed for the model and for each of the human experts.

### Occlusion Test

Similarly to Lee et al, 2016, an occlusion test was performed to identify the areas contributing the most to the neural network's assignment of the predicted diagnosis. A blank 20×20 pixel box was systematically moved across every possible position in the image and the probabilities of the disease were recorded. The highest drop in the probability represents the region of interest that contributed the highest importance to the deep learning algorithm.

### Demonstrative Website and Tool

The publicly available trained model at https://www.medfirstview.com allows interested parties to test the classification performance of the model. The Python tool implements a Tkinter user interface that allows a user to upload an image, loads the trained model, and streams the image through the neural network to make a classification. The website uses the same method without the Tkinter user interface.

### Quantification and Statistical Analysis

### Data and Software Availability

All deep learning methods were implemented using either TensorFlow (https://www.tensorflow.org) or PyTorch (https://www.pytorch.org). ImageNet public database of images can be found at https://www.image-net.org. Image data was translated into high resolution TIFF images using a proprietary Heidelberg script to extract B-scans and the 3 most foveal cuts of volume scans. All other scripts and analysis for this project were created by the researchers of this project written in Python and Bash.

### Application to Non-Lung Diseases

Although described with respect to lung diseases, it is also contemplated herein that the method, system, and/or computer-readable medium in the present disclosure can be used in diagnosis of non-lung diseases or conditions.

We claim:

1. A method for providing a medical diagnosis, comprising:
a) obtaining a medical image of a lung;
b) evaluating the medical image using a predictive model trained using a machine learning procedure; and
c) determining, by the predictive model, whether or not the medical image is indicative of a disease or disorder of the lung, the determination having a sensitivity greater than 90% and a specificity greater than 90%.

2. The method of claim 1, wherein the machine learning procedure comprises a deep learning procedure.

3. The method of claim 1 or 2, wherein the machine learning procedure comprises a convolutional neural network.

4. The method of any one of claims 1-3, further comprising subjecting the medical image of the lung to an image occlusion procedure.

5. The method of any one of claims 1-4, wherein the machine learning procedure comprises a transfer learning procedure.

6. The method of claim 5, wherein the transfer learning procedure comprises pre-training the machine learning procedure using non-medical or unlabeled medical images obtained from a large image dataset to obtain a pre-trained model.

7. The method of claim 6, wherein the transfer learning procedure further comprises training the pre-trained model using a set of medical images that is smaller than the large image dataset.

8. The method of any one of claims 1-7, further comprising making a medical treatment recommendation based on the determination.

9. The method of any one of claims 1-8, wherein the medical image of the lung is a chest X-ray.

10. The method of any one of claims 1-9, wherein the medical image comprises an X-ray image.

11. The method of any one of claims 1-10, wherein the medical image comprises a lung X-ray.

12. The method of any one of claims 1-11, wherein the disease or disorder of the lung is selected from the group consisting of: pneumonia, childhood pneumonia, emphysema, tuberculosis, and lung cancer.

13. A non-transitory computer-readable medium comprising machine-executable code that, upon execution by one or more computer processors, implements a method for providing a medical diagnosis, the method comprising:
a) obtaining a medical image of a lung;
b) evaluating the medical image using a predictive model trained using a machine learning procedure; and
c) determining, by the predictive model, whether or not the medical image is indicative of a disease or disorder of the lung, the determination having a sensitivity greater than 90% and a specificity greater than 90%.

14. The non-transitory computer-readable medium of claim 13, wherein the machine learning procedure comprises a deep learning procedure.

15. The non-transitory computer-readable medium of claim 13 or 14, wherein the machine learning procedure comprises a convolutional neural network.

16. The non-transitory computer-readable medium of any one of claims 13-15, wherein the method further comprises subjecting the medical image of the lung to an image occlusion procedure.

17. The non-transitory computer-readable medium of any one of claims 13-16, wherein the machine learning procedure comprises a transfer learning procedure.

18. The non-transitory computer-readable medium of claim 17, wherein the transfer learning procedure comprises pre-training the machine learning procedure using non-medical or unlabeled medical images obtained from a large image dataset to obtain a pre-trained model.

19. The non-transitory computer-readable medium of claim 18, wherein the transfer learning procedure further comprises training the pre-trained model using a set of medical images that is smaller than the large image dataset.

20. The non-transitory computer-readable medium of any one of claims 13-19, wherein the method further comprises making a medical treatment recommendation based on the determination.

21. The non-transitory computer-readable medium of any one of claims 13-20, wherein the medical image of the lung is a chest X-ray.

22. The non-transitory computer-readable medium of any one of claims 13-21, wherein the medical image comprises an X-ray image.

23. The non-transitory computer-readable medium of any one of claims 13-22, wherein the medical image comprises a plurality of lung X-rays.

24. The non-transitory computer-readable medium of any one of claims 13-23, wherein the disease or disorder of the lung is selected from the group consisting of: pneumonia, childhood pneumonia, emphysema, tuberculosis, and lung cancer.

25. A computer-implemented system comprising: a digital processing device comprising: at least one processor, an operating system configured to perform executable instructions, a memory, and a computer program including instructions executable by the digital processing device to create an application for providing a medical diagnosis of a disease or disorder or a lung, the application comprising:
a) a software module for obtaining a medical image of a lung;
b) a software module for analyzing the medical image using a predictive model trained using a machine learning procedure; and
c) a software module for determining, by the predictive model, whether or not the medical image of the lung is indicative of a disease or disorder of the lung, the determination having a sensitivity greater than 90% and a specificity greater than 90%.

26. The system of claim 25, wherein the machine learning procedure comprises a deep learning procedure.

27. The system of claim 25 or 26, wherein the machine learning procedure comprises a convolutional neural network.

28. The system of any one of claims 25-27, wherein the application further comprises a software module for subjecting the medical image of the lung to an image occlusion procedure.

29. The system of any one of claims 25-28, wherein the machine learning procedure comprises a transfer learning procedure.

30. The system of claim 29, wherein the transfer learning procedure comprises pre-training the machine learning procedure using non-domain medical images obtained from a large image dataset to obtain a pre-trained model.

31. The system of claim 30, wherein the transfer learning procedure further comprises training the pre-trained model using a set of labeled medical images that is smaller than the large image dataset.

32. The system of any one of claims 25-31, wherein the application further comprises a software module for making a medical treatment recommendation based on the determination.

33. The system of any one of claims 25-32, wherein the medical image of the lung is a chest X-ray.

34. The system of any one of claims 25-33, wherein the medical image comprises an X-ray image.

35. The system of any one of claims 25-34, wherein the medical image comprises a plurality of lung X-rays.

36. The system of any one of claims 25-35, wherein the disease or disorder of the lung is selected from the group consisting of: pneumonia, childhood pneumonia, emphysema, tuberculosis, and lung cancer.
